(19) United States
(12) Patent Application Publication
(10) Pub. No.: US 2012/0058193 A1
(43) Pub. Date: Mar. 8, 2012

(54) INJECTABLE SMART GEL AND METHOD FOR FABRICATING THE SAME

(76) Inventors: Dean-Mo LIU, Hsinchu County (TW); Meng-Hsuan Hsiao, New Taipei City (TW); Li Jie Lin, New Taipei City (TW)

(21) Appl. No.: 13/079,439

(22) Filed: Apr. 4, 2011

(30) Foreign Application Priority Data
Sep. 7, 2010 (TW) 099130148

(51) Int. Cl. A61K 9/51; A61K 31/4015; A61P 25/08; A61K 47/36

(52) U.S. Cl. 424/493; 514/777; 424/490; 514/425

(57) ABSTRACT

An injectable smart gel and a method for fabricating the same are disclosed. A basic structural stabilizer/polymeric electrolyte and a diluting solution are added to a modified chitosan to regulate the chitosan solution to have a pH value closing to that of the human body and form a flowable chitosan sol. The flowable chitosan sols formed thereby are respectively converted into inflowable chitosan gels via increasing the temperature thereof to the human body temperature, and via adding calcium ion or regulating the chitosan sol into an acidic solution. The injectable smart gel fabricated thereby is injectable and able to function as a carrier of magnetism-sensitive medicine-containing nanocapsules. The medicine can be released to the injectable smart gel with an external non-contact force, such as a magnetic field, an electric field or an ultrasonic wave, for long-acting and multi-stage medicine delivery. The present invention is very useful in biomedical engineering.

### BACKGROUND OF THE INVENTION

1. Field of the Invention

The present invention relates to an injectable gel and a method for fabricating the same, particularly to an injectable smart gel, which is injected in vivo to function as a medicine carrier able to perform long-acting and multi-stage medicine delivery, and a method for fabricating the same.

2. Description of the Related Art

General injectable gels (such as hyaluronic acid) are expensive but have limited applications, primarily functioning as humectant and filling of esthetic surgery. In bioengineering, chitosan is also fabricated into a gel only functioning as a cell carrier to repair bones or a human body lubricant.

Chitosan is derived from chitin. However, the solubility performance of chitosan has greatly expanded the application field of chitin. Recently, the concerned fields have paid much attention on chitosan-based biomaterials and developed many products thereof. Chitosan is hard to absorb water or dissolve in water. Chitosan does not form a solid gel but precipitates when the pH value thereof approaches neutrality. In such a case, the biomedical application of chitosan is pretty limited. So far, some researches have been devoted to the application of injectable chitosan to a medicine carrier. However, all of them are addressed to unmodified chitosan. Those researches neither promote performance of chitosan nor achieve controllable medicine delivery with chitosan. In other words, the conventional medicine delivery technologies of chitosan are unlikely to achieve timely control or inhibition of a disease.

### SUMMARY OF THE INVENTION

The primary objective of the present invention is to provide an injectable smart gel and a method for fabricating the same, wherein chitosan is modified to have thermosensitivity, whereby the modified chitosan is injectable at a lower temperature and solidifies to form a chitosan gel at the human body temperature. The injectable smart gel of the present invention can incorporate with medicine-containing nanocapsules sensitive to a physical factor, whereby an external physical factor, such as a magnetic field, an electric field or an ultrasonic wave, can control the release of medicine in real time, and whereby the patients are greatly convenienced in therapy and living.

Another objective of the present invention is to provide an injectable smart gel and a method for fabricating the same, wherein a polymeric electrolyte (such as an alginate) is added to the modified chitosan to form a flowable chitosan sol, and wherein the chitosan sol is converted into a chitosan solid gel via adding calcium ion or regulating the chitosan sol into an acidic solution, whereby is achieved the injectability and sol-gel conversion of chitosan. The injectable smart gel of the present invention can incorporate with medicine-containing nanocapsules sensitive to a physical factor, whereby an external physical factor, such as a magnetic field, an electric field or an ultrasonic wave, can control the release of medicine in real time, and whereby the patients are greatly convenienced in therapy and living.

To achieve the abovementioned objectives, the present invention proposes a method for fabricating an injectable smart gel, which comprises steps: providing a hydrophobically-modified or amphiphilically-modified chitosan solution with a concentration of 0.1-10% (w/v); at a temperature of 4-20° C., adding 0.1-10% (w/v) of a basic structural stabilizer and 80-99.5% (w/v) of a diluting solution to the modified chitosan solution to regulate the pH value of the modified chitosan solution and convert the modified chitosan solution into a chitosan sol having a near-neutrality pH value; and converting the chitosan sol into a solid chitosan gel via increasing the temperature of the chitosan sol to 30-40° C.

The injectable smart gel fabricated according to the method of the present invention has a pH value similar to that of a human body and is injectable because of the thermosensitivity thereof. The injectable smart chitosan gel is in form of a flowable sol in a lower temperature and in form of an almost unflowable chitosan gel at a higher temperature. The injectable smart gel is mixed with magnetism-sensitive medicine-containing nanocapsules to form a medical element able to undertake long-acting and multi-stage medicine delivery with a non-contact force. The injectable smart gel can be placed in a human body without using a surgical operation. Further, the injectable smart gel needn't be removed with a surgical operation because of the biodegradability thereof. Therefore, patients are exempted from double surgical operations that they have to suffer in the conventional technology. Accordingly, the injectable smart gel fabricated according to the method will be very useful in many fields, such as bioengineering, chronic diseases, and esthetic medicine.

The present invention also proposes another method for fabricating an injectable smart gel, which comprises steps: providing a hydrophobically-modified or amphiphilically-modified chitosan solution with a concentration of 0.1-10% (w/v); adding 0.1-10% (w/v) of a polymeric electrolyte and 80-99.5% (w/v) of a diluting solution to the modified chitosan solution to regulate the pH value of the modified chitosan solution and form a near-neutrality chitosan sol; adding calcium ion to the chitosan sol or regulating the chitosan sol into an acidic solution to form a chitosan solid gel. Different from the conventional chitosan-alginate mixture that becomes gel instantly after mixing, the product fabricated according to the method of the present invention remains injectable. Further, the injectable smart gel fabricated according to the method can incorporate with magnetism-sensitive medicine-containing nanocapsules to form a medical element able to undertake long-acting and multi-stage medicine delivery with a non-contact force. The storage modulus of the smart gel is similar to that of the muscle. The smart gel more corresponds to the muscle tissue. When the nano gel structure is harmed due to the movement or other behavior of a human body, the gel structure can restore itself to maintain original medicine delivery mechanism. The gel is not easily affected by activities of the human body. Therefore, the injectable smart gel fabricated according to the method will be very useful in many fields, such as bioengineering, chronic diseases, and esthetic medicine.

Below, the embodiments are described in detail in cooperation with the drawings to make easily understood the objectives, characteristics and accomplishments of the present invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flowchart of a method for fabricating an injectable smart gel according to one embodiment of the present invention;

FIG. 2 is a flowchart of a method for fabricating an injectable smart gel according to another embodiment of the present invention;

FIG. 3 shows the accumulated release ratio of a medicine released from an injectable smart gel multi-stage stimulated by an alternating magnetic field at a frequency of 40.1 kHz and detected by an ultraviolet-visible light spectrometer;

FIG. 4 is a rheological diagram of an injectable smart gel according to an embodiment of the present invention; and

FIG. 5A-5C are pictures of an injectable smart gel for self-reconstruction according to an embodiment of the present invention.

### DETAILED DESCRIPTION OF THE INVENTION

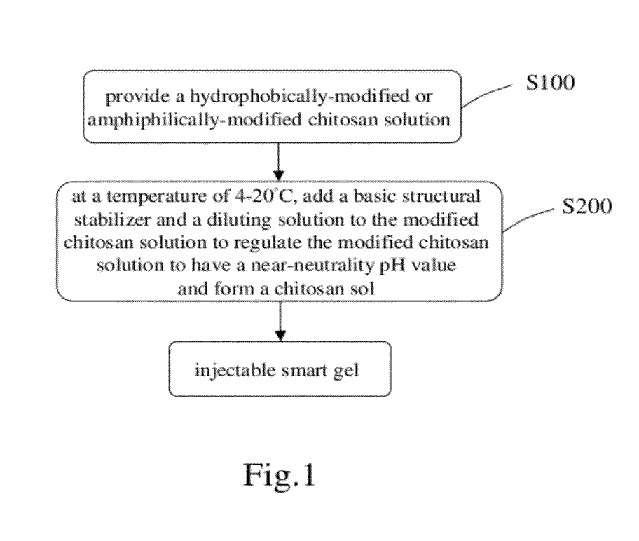

Refer to FIG. 1 a flowchart of a method for fabricating an injectable smart gel according to one embodiment of the present invention.

In Step S100, provide a hydrophobically-modified or amphiphilically-modified chitosan solution with a concentration of 0.1-10% (w/v). In one embodiment, a powder of 95% deacetylated chitosan with a molecular weight of 50 kDa-250 kDa is hydrophilically modified with a haloacetic acid or hydrophobically modified with an acid anhydride having a long chain with 2-12 carbon atoms. The haloacetic acid is selected from a group consisting of monochloracetic acid, dichloracetic acid, trichloracetic acid, monobromoacetic acid, dibromoacetic acid, and bromochloroacetic acid. The acid anhydride may be acetic anhydride or hexanoic anhydride. The modified chitosan solution has negative zeta potential, biodegradability and self-assemble capability to form nanocapsules.

In Step S200, at a temperature of 4-20° C., add 0.1-10% (w/v) of a basic structural stabilizer and 80-99.5% (w/v) of a diluting solution to the modified chitosan solution. The basic structural stabilizer is selected from a group consisting of β-glyceryl sodium phosphate, sodium acid carbonate, genipin (a natural crosslinker) and a combination thereof. The solution may be water or a mixture of water and an organic solvent (an oil-like compound). In the case of water and an organic solvent, 1-20% of an organic solvent is added to the 80-99.5% (w/v) of diluting solution. The organic solvent is selected from a group consisting of DMSO (dimethyl sulfoxide), alcohol, glycol and glycerin.

Thereby, the modified chitosan solution is regulated to have a pH value of 5-9, which is similar to the pH value of the human body, to form a thermosensitive injectable smart gel. The injectable smart gel is in form of a flowable chitosan sol having injectability at a lower temperature of 4-20° C., and in form of an inflowable (solid) chitosan gel when the temperature rises to 30-40° C.

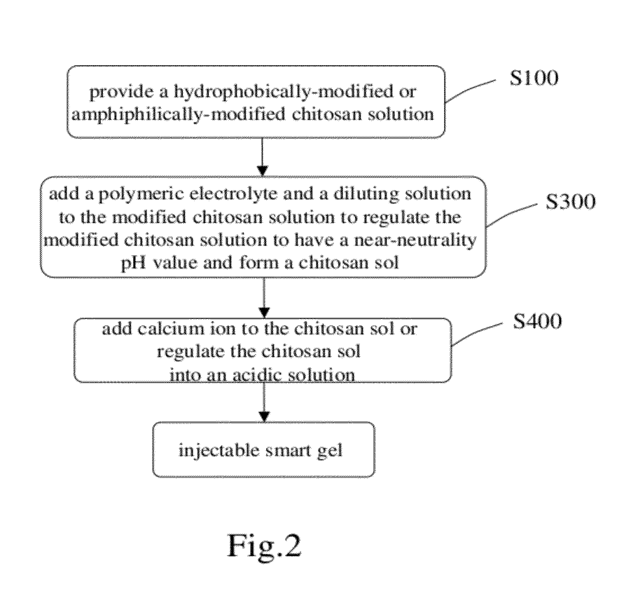

Refer to FIG. 2 a flowchart of a method for fabricating an injectable smart gel according to another embodiment of the present invention.

In Step S100, provide a hydrophobically-modified or amphiphilically-modified chitosan solution with a concentration of 0.1-10% (w/v). In Step S300, add 0.1-10% (w/v) of a polymeric electrolyte (such as an alginate) and 80-99.5% (w/v) of a diluting solution to the modified chitosan solution. The polymeric electrolyte is negatively-charged, biodegradable and soluble in a neutral environment. When the polymeric electrolyte is mixed with the chitosan solution, the mixture solution neither precipitates nor forms a solid gel. In Step S300, the solution may be water or a mixture of water and an organic solvent. In the case of water and an organic solvent, 1-20% of an organic solvent is added to the 80-99.5% (w/v) of diluting solution. The organic solvent is selected from a group consisting of DMSO (dimethyl sulfoxide), alcohol, glycol and glycerin. Thereby, the modified chitosan solution is regulated to have a pH value of 5-9, which is similar to the pH value of the human body, to form a flowable chitosan sol—an injectable smart gel.

In Step S400, add calcium ion to the chitosan sol or modify the chitosan sol into an acidic solution to form an inflowable (solid) chitosan gel.

Below is described in detail one embodiment of the method for fabricating an injectable smart gel.

The process of synthesizing an amphiphilically-modified chitosan (CHC) powder includes steps:
1. Place 20 g chitosan in a 1000 ml three-necked round-bottomed flask, and add 200 ml isopropanol to the chitosan, and agitate the mixture for 30 minutes to form a suspension.
2. Add 5 ml 13.3N sodium hydroxide solution to the suspension each 5 minutes, totally 10 times and 50 ml.
3. Agitate the solution for 30 minutes; add 5 parts of chloroacetic acid into the flask totally 100 g within 5 minutes. Note: chloroacetic acid should be added gradually lest it cannot dissolve fully.
4. Heat the solution to a temperature of 60° C. for 4 hours with an oil bath; collect the reaction product with a suction filtering method, and use a liquid having water and alcohol by a ratio of 1:9 to flush the product during filtering.
5. Dry the product in an oven at a temperature of 60° C. for one day to obtain a water-soluble pale-yellow NOCO (N, O-carboxymethyl chitosan) powder.
6. Place 4 g NOCO in a 250 ml reaction bottle, and add 100 ml pure water into the bottle, and agitate the mixture for one day to make the mixture dissolve fully.
7. Add 50 ml methanol to the solution and agitate the mixture solution uniformly; add 2.8 ml hexanoic anhydride to the solution to undertake a reaction for 24 hours.
8. Collect the reaction product with a dialysis membrane, wherein the product is dialyzed with a liquid containing water and alcohol by a ratio of 1:4 for one day and then dialyzed with pure alcohol for 2 days to remove acid and ions.
9. Dry the collected product at a temperature of 60° C. for one day to obtain an amphiphilically-modified chitosan powder.

Next, fabricate an injectable smart gel. Herein are introduced two embodiments: (a) the fabrication of an amphiphilically-modified chitosan (CHC) gel, and (b) the fabrication of a CHC/alginate gel.
(a) The fabrication of an amphiphilically-modified chitosan (CHC) gel:

In this embodiment, β-glyceryl sodium phosphate having a negative zeta potential and a basic pH value is used to regulate the injectable smart gel to have a neutral pH value that is close to the human body pH value 7.4. Each β-glyceryl sodium phosphate molecule has several hydroxyls, and the hydrogen bonds between hydroxyls and chitosan molecules can stabilize the gel structure. Besides, the cross-linking agent, such as β-glyceryl sodium phosphate or genipin, will cross link with amphiphilically-modified chitosan molecules at the human body temperature, which can prevent the gel structure from being permeated and loosened by water in an aqueous environment. Thereby, the physical properties of chitosan gel can be maintained for a long time in an aqueous environment. The fabrication process thereof comprises the following steps:
1. Dissolve 0.5-3 g CHC powder in 100 ml of deionized water, PSB (Phosphate Buffer Solution) or SBF (Simulated Body Fluid) to form a 0.5-3% (w/v) CHC solution.
2. Add glycerol to the CHC solution to form a CHC solution containing 0-10% (w/v) glycerol.
3. At a temperature lower than 4° C., add 0.1-1 g β-glyceryl sodium phosphate to 10 ml CHC/glycerol solution to form a CHC/glycerol solution containing 1-10% (w/v) β-glyceryl sodium phosphate and having a pH value of 6.5-7.4. The CHC/glycerol/β-glyceryl sodium phosphate-containing solution is in form of a flowable sol at a lower temperature of 4-20° C., and is converted into an inflowable gel when the temperature rises to 30-40° C.
(b) The fabrication of a CHC/alginate gel:

The fabrication process thereof comprises the following steps:
1. Respectively dissolve 2-4 g sodium alginate in 100 ml pure water.
2. Dissolve 2 g CHC powder in 100 ml pure water to form a 2% (w/v) CHC solution.
3. Add 100 μl NaOH solution having a pH value=11 to 10 ml 2% (w/v) CHC solution to regulate the CHC solution to have a slight basicity.
4. Respectively add 1 ml glycerol into 2%, 3% and 4% sodium alginate solutions.
5. Mix the CHC solution with the sodium alginate solutions by ratios of 1:1 and 1:2 to form solutions.
6. Add calcium ion to the solutions, or regulate the solutions to have acidity, to form inflowable gels.

After fabrication, the injectable smart gel can further incorporate with magnetism (or another physical factor)-sensitive medicine-containing nanocapsules, and the medicine will be magnetically, electrically or ultrasonically controlled to release. Below, the combination of the CHC gel and magnetism-sensitive medicine-containing nanocapsules is used as an exemplification.

Below is described the process of fabricating magnetism-sensitive silica-shell nanocapsules. Firstly, dissolve ferric oxide nanoparticles and a medicine in dichloromethane. Next, mix lipophilic dichloromethane with an aqueous solution of hydrophilic polyvinyl alcohol via a microemulsion method. Polyvinyl alcohol is a surfactant. After ultrasonic vibration for 180 seconds, oil-in-water nanocapsules are formed in the microemulsion liquid. Next, add TEOS (tetraethoxysilane) to the oil-in-water nanocapsules. Next, hydrolyze and condensate silica with a sol-gel method to wrap the ferric oxide nanocapsules; alternatively, wrap the ferric oxide nanocapsules with a chitin having a negative zeta potential in an acidic environment. Thus are achieved magnetism-sensitive nanocapsules with a silica, chitin or silica/chitin shell and a core containing ferric oxide and polyvinyl alcohol. The nanocapsules has a diameter of about 50-200 nm, and the medicine contained thereinside may be an anti-cancer medicine, a peptide or a protein.

The magnetism-sensitive nanocapsules are added to the chitosan sol by an appropriate amount and mixed with the chitosan sol uniformly via simple mechanical agitation. Next, at a lower temperature (4-20° C.), the acidic modified chitosan sol is regulated to near the neutral pH value of the human body so as to increase the biocompatibility of the injectable smart gel to the human body. The modified chitosan transforms from sol into gel when the temperature rises to 37° C. Therefore, the modified chitosan sol becomes a neutral solid gel when injected into the human body. Then, an external magnetic field is used to control the magnetism-sensitive nanocapsules to release medicine to the injectable smart gel, and the injectable smart gel further releases medicine to the human body. Thereby is achieved the objective of long-acting and multi-stage medicine delivery.

Experiments are used to verify the performance of the injectable smart gel functioning as a medicine carrier injected into an animal body and releasing medicine to the animal body, wherein a small-molecule anti-epilepsy medicine ESM (ethosuximide) is used in the experiments. In the process of synthesizing the magnetism-sensitive nanocapsules, the medicine molecules are dissolved in lipophilic dichloromethane. Then, the medicine is wrapped in the oil-in-water ferric oxide (magnetism-sensitive) nanocapsules. An ultraviolet-visible light spectrometer is used to examine the release of the small-molecule medicine.

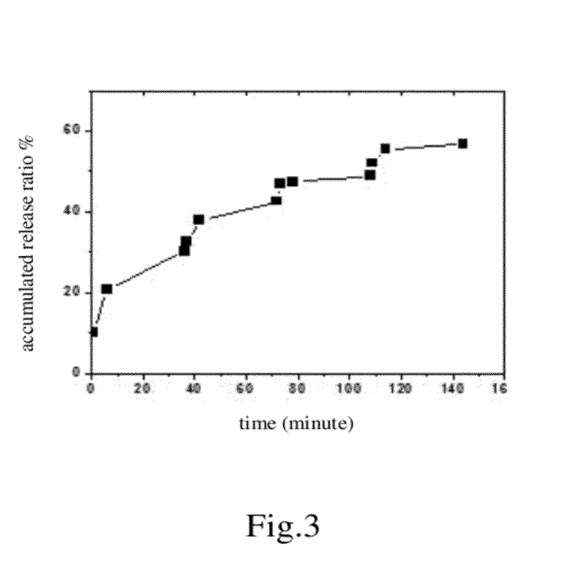

The quantity of the released medicine is determined by the intensity of the wavelength absorbed by the medicine in deionized water. ESM absorbs a wavelength of 258 nm. In the experiment, 2 ml injectable smart gel is processed in a 15 ml centrifugal tube and then placed in 8 ml deionized water. The injectable smart gel is stimulated by an alternating magnetic field each 35 minutes. FIG. 3 shows the experiment results of the injectable smart gel not stimulated by a magnetic field and the injectable smart gel multi-stage stimulated by a magnetic field. FIG. 3 proves that the injectable smart gel can function as a medicine-delivery element. The wrapped medicine is controlled to deliver to the injectable smart gel by a magnetic field, and the injectable smart gel further gradually releases the medicine out. Therefore, the injectable smart gel of the present invention can apply to various medicines and many fields.

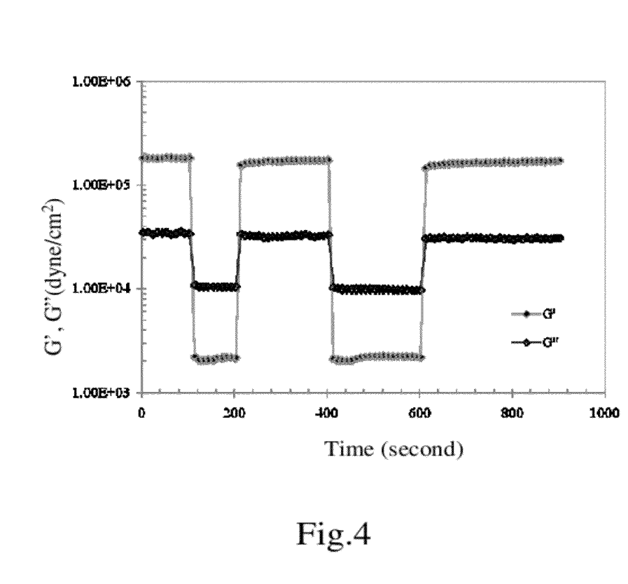

When the strain is exerted to the injectable smart gel, the injectable smart gel is deformed, and then self-reconstructed. In order to understand the self-reconstruction situation, refer to FIG. 4. G′ represents the coagulability of the gel, and G″ represents the flowability of the gel. When G′ is larger than G″, the smart gel is a coagulation gel. However, when the quite strain (deformation force) is exerted to the smart gel, the smart gel becomes a sol gel with the flowability and G′ is smaller than G″. As a result, the sol gel has the properties of the formability.

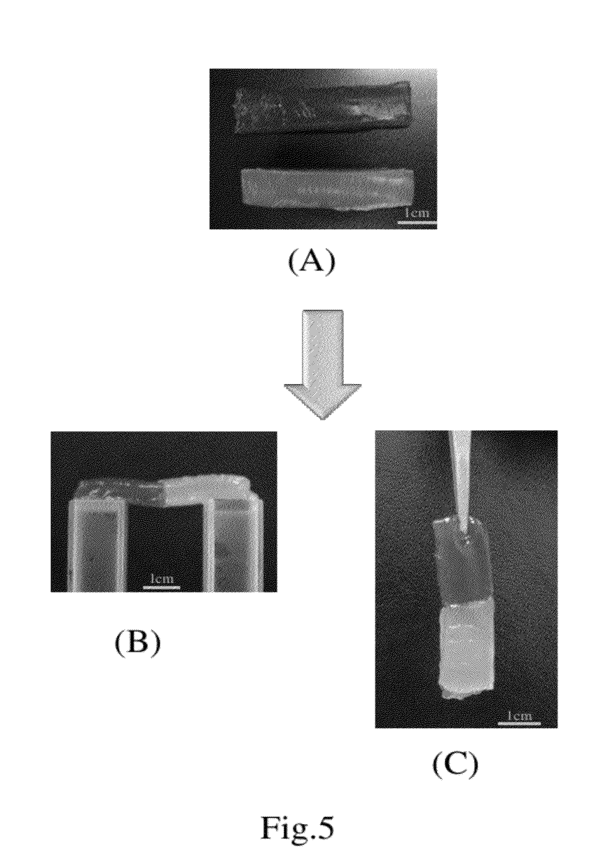

FIG. 5A shows the two separate injectable smart gels. FIG. 5B shows the two separate injectable smart gels connected with each other. FIG. 5C shows that two separate injectable smart gels are combined into an injectable smart gel. In other words, a skilled person in the art can exert an external force to the injectable smart gel to obtain the wanted shape. Alternatively, the two separate gels can be combined with each other to obtain the required volume. Since the additive gel is combined with original gel completely, the injectable smart gel has the self-reconstruction properties.

Via modifying chitosan, the injectable smart gel not only has injectability but also has better water-retention capability than the conventional chitosan gel. The injectable smart gel can incorporate with magnetism-sensitive nanocapsules and function as a medicine storage element, whereby nanocapsules or medicine molecule can be retained in the human body and released in multiple stages during a period of time via externally applying a magnetic field, electric field or ultrasonic wave. The uniqueness of the present invention is very favorable for the therapy of chronic patients. Being a medicine storage/delivery element, the injectable smart gel can be applied to a patient via oral intake, subcutaneous injection, intramuscular injection, rectal injection, or peritoneal injection.

Therefore, the injectable smart gel can be disposed inside the human body without surgery. Further, the injectable smart gel is biodegradable in the human body. The biodegrade of the injectable smart gel can occur spontaneous, but the speed of biodegrade is externally controllable. Thus, the present invention can exempt patients from suffering surgical operations twice. Accordingly, the injectable smart gel fabricated according to the method will be very useful in many fields, such as bioengineering, chronic diseases, and esthetic medicine.

The embodiments described above are only to exemplify the present invention but not to limit the scope of the present invention. Any equivalent modification or variation according to the spirit of the present invention is to be also included within the scope of the present invention, which is based on the claims stated below.

What is claimed is:

1. A method for fabricating an injectable smart gel, comprising steps:
providing a hydrophobically-modified or amphiphilically-modified chitosan solution with a concentration of 0.1-10% (w/v); and
at a temperature of 4-20° C., adding 0.1-10% (w/v) of a basic structural stabilizer and 80-99.5% (w/v) of a diluting solution to said modified chitosan solution to regulate said modified chitosan solution to have a pH value of 5-9 and form a near-neutrality chitosan sol, which becomes a solid chitosan gel when temperature rises to 30-40° C.

2. The method for fabricating an injectable smart gel according to claim 1, wherein said diluting solution is water or a mixture of water and an oil-like compound, and wherein said oil-like compound is selected from a group consisting of DMSO (dimethyl sulfoxide), alcohol, glycol and glycerin.

3. The method for fabricating an injectable smart gel according to claim 1, wherein said basic structural stabilizer is selected from a group consisting of genipin, β-glyceryl sodium phosphate, sodium acid carbonate, and a combination thereof.

4. The method for fabricating an injectable smart gel according to claim 1, wherein said chitosan solution is made from a powder of 95% deacetylated chitosan with a molecular weight of 50 kDa-250 kDa.

5. The method for fabricating an injectable smart gel according to claim 1, wherein said chitosan solution is hydrophilically modified with a haloacetic acid, and wherein said haloacetic acid is selected from a group consisting of monochloracetic acid, dichloracetic acid, trichloracetic acid, monobromoacetic acid, dibromoacetic acid, and bromochloroacetic acid.

6. The method for fabricating an injectable smart gel according to claim 1, wherein said chitosan solution is hydrophobically modified with an acid anhydride having a long chain with 2-12 carbon atoms, and wherein said acid anhydride is acetic anhydride or hexanoic anhydride.

7. The method for fabricating an injectable smart gel according to claim 1 further comprising a step of mixing said chitosan sol with magnetism-sensitive nanocapsules containing medicine molecules.

8. The method for fabricating an injectable smart gel according to claim 7, wherein a ferric oxide-containing core wraps said medicine molecules, and wherein said core is coated with a shell, and wherein said shell is made from silica or chitin, and wherein said core is a composite structure containing ferric oxide and polyvinyl alcohol, and wherein said medicine molecule is an anti-cancer medicine, a peptide or a protein.

9. The method for fabricating an injectable smart gel according to claim 7, wherein said magnetism-sensitive nanocapsules have a diameter of 50-200 nm.

10. An injectable smart gel fabricated according to claim 1.

11. A method for fabricating an injectable smart gel, comprising steps:
providing a hydrophobically-modified or amphiphilically-modified chitosan solution with a concentration of 0.1-10% (w/v);
adding 0.1-10% (w/v) of a polymeric electrolyte and 80-99.5% (w/v) of a diluting solution to said modified chitosan solution to regulate said modified chitosan solution to have a pH value of 5-9 and form a near-neutrality chitosan sol; and
adding calcium ion to said chitosan sol or regulating said chitosan sol into an acidic solution to form a chitosan solid gel.

12. The method for fabricating an injectable smart gel according to claim 11, wherein said polymeric electrolyte is an alginate.

13. The method for fabricating an injectable smart gel according to claim 11, wherein said diluting solution is water or a mixture of water and an oil-like compound, and wherein said oil-like compound is selected from a group consisting of DMSO (dimethyl sulfoxide), alcohol, glycol and glycerin.

14. The method for fabricating an injectable smart gel according to claim 11, wherein said chitosan solution is made from a powder of 95% deacetylated chitosan with a molecular weight of 50 kDa-250 kDa.

15. The method for fabricating an injectable smart gel according to claim 11, wherein said chitosan solution is hydrophilically modified with a haloacetic acid, and wherein said haloacetic acid is selected from a group consisting of monochloracetic acid, dichloracetic acid, trichloracetic acid, monobromoacetic acid, dibromoacetic acid, and bromochloroacetic acid.

16. The method for fabricating an injectable smart gel according to claim 11, wherein said chitosan solution is hydrophobically modified with an acid anhydride having a long chain with 2-12 carbon atoms, and wherein said acid anhydride is acetic anhydride or hexanoic anhydride.

17. The method for fabricating an injectable smart gel according to claim 11 further comprising a step of mixing said chitosan sol with magnetism-sensitive nanocapsules containing medicine molecules.

18. The method for fabricating an injectable smart gel according to claim 17, wherein a ferric oxide-containing core wraps said medicine molecules, and wherein said core is coated with a shell, and wherein said shell is made from silica or chitin, and wherein said core is a composite structure containing ferric oxide and polyvinyl alcohol, and wherein said medicine molecule is an anti-cancer medicine, a peptide or a protein.

19. The method for fabricating an injectable smart gel according to claim 17, wherein said magnetism-sensitive nanocapsules have a diameter of 50-200 nm.

20. An injectable smart gel fabricated according to claim 11.
